(19) United States
(12) Patent Application Publication
(10) Pub. No.: US 2004/0133449 A1
(43) Pub. Date: Jul. 8, 2004

(54) Method and system for facilitating drug development

(76) Inventor: Alpheus Bingham, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/333,143

(22) PCT Filed: Jul. 20, 2001
(86) PCT No.: PCT/US01/21107

(51) Int. Cl. G06F 17/60; A01N 25/00; A61K 47/00

(52) U.S. Cl. 705/2; 514/789

(57) ABSTRACT

The present invention relates to methods and systems for facilitating medical product development by involving a plurality of developers in the development process and distributing equity to the developers based upon the level and nature of their contributions. The invention further relates to use of a computer network, such as the Internet, in the development of drugs.

### BACKGROUND OF THE INVENTION

The present invention relates to methods and systems for facilitating medical product development by involving a plurality of developers in the development process and distributing equity to the developers based upon the level and nature of their contributions. The invention further relates to use of a computer network, such as the Internet, in the development of drugs.

The complete development process for a drug includes many steps which may involve substantial amounts of time, resources and expense. Drug development begins at its earliest stage with the synthesis or acquisition of a molecule, and identification of the molecule for possible therapeutic or other useful indications. Typically, safety and efficacy of the potential drug are then explored. Formal requirements for drug approval include verifying synthesis routes, conducting clinical trials, making regulatory submissions, and finally preparing the drug for market. Each of these steps requires specific expertise, and the time and expense involved can be great. Development of drugs can therefore be a very expensive, high risk business.

Consequently, it would be desirable to have a way to distribute drug development among a plurality of different companies. This would allow the burdens and risks of drug development to be shared, along with the financial rewards. It would also be advantageous to be able to utilize diverse talents and expertise available in different companies, and similarly to utilize these resources more efficiently. Involving multiple developers would enable the sharing of the collective ideas and expertise of diverse and widely placed resources involved throughout the development process.

Ultimately, distributing the development process would lead to the development and marketing of drugs that otherwise may not have been possible due to unfavorable economics, limited technical expertise, or limited resources for a given company. Also, development projects can frequently be more rapidly evaluated through the involvement of multiple developers. Accordingly, drugs can be developed at a lower overall cost, thereby favorably impacting profitability and assisting in the reduction of drug prices.

The present invention involves methods and systems that satisfy the foregoing needs and advantages by providing for the involvement of multiple companies in the drug development process in a unique way. Development activities are distributed among a plurality of companies which are compensated for their efforts out of subsequent drug revenues.

### SUMMARY OF THE INVENTION

One embodiment of the present invention relates to a method and system to facilitate the development of drugs by the participation of more than one developer. In one form of this embodiment, values are assigned to various development activities, and the participating developers receive the corresponding equity in the drug for completing the identified activities.

In one aspect of the present invention, a method to facilitate development of a drug includes publishing a description of a development project for the drug that has a number of different development activities and inviting participation in the development project by any interested party. An outside developer completes one the development activities and is provided an interest in the development project.

In a further aspect, a method of medical product development comprises publishing a medical product development project description that includes a number of development activities and an equity amount for each of the development activities. In response to this publication an outside developer performs one of the activities and equity is assigned to the outside developer in accordance with the equity amount corresponding to the one of the development activities.

In another aspect, the invention provides a method that comprises assigning equity values to different development activities and publishing these values to potential outside developers, receiving a communication from a developer regarding at least one of the activities, having the activity completed by the developer, and assigning to the developer the equity value corresponding to the completed activity. In a related aspect, the invention includes compensating the developer based upon application of the assigned equity value against drug revenues following launch of the drug.

Still another aspect of the present invention includes a method of medical product development which comprises receiving a published description of a medical product development project over a computer network, performing one or more development activities for the project in response to this description, and receiving equity in the project for performing such activities. The description can provide a number of development activities and an invitation to perform one or more of these activities.

In yet a further aspect of the present invention, a method of medical product development includes reviewing a published description of a medical product development project from a development initiator that includes a number of development activities and an equity amount for each of these activities. This method also includes communicating with the development initiator in response to the published description and completing one or more of the development activities. Equity is received in accordance with the equity amount for the one or more development activities completed.

Various unique apparatus, devices and systems for accomplishing the methods of the present invention are also provided.

Other aspects, embodiments, forms, features, and objects of the present invention shall become apparent from the drawings and description contained herein.

### BRIEF DESCRIPTION OF THE DRAWINGS

FIG. 1 is a schematic view of a system of one embodiment of the present invention.

FIG. 2 is a development project description for the system of FIG. 1.

### DETAILED DESCRIPTION OF PREFERRED EMBODIMENTS OF THE INVENTION

While the present invention may be embodied in many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The present invention facilitates the development of drugs through various stages by involving a plurality of developers. The method comprises the assignment of equity values to different development activities, and completing the development activities by a plurality of developers that thereby acquire the respective equity values in the developed drug. In one aspect, the equity values are determined based upon a distribution scheme that allocates equity points to each of the development activities. Allocated equity can be used to allocate revenues upon the sale of the developed drug. This method is advantageous in distributing the risks and rewards of drug development over a plurality of companies. Development occurs more quickly and with less risk to individual companies, and companies can be involved that otherwise would not have had the ability to pursue drug development on their own.

The present invention assists in the development process in several respects. A company can facilitate its own development of a drug through the involvement of an outside developer in at least one development activity. Alternatively, several companies can collaborate on the development of a drug or compete with one another to complete one or more drug development stages. “Virtual drug development” can be accomplished by a coordinating company that brings together multiple developers to perform the various development activities required to bring a drug to market. In each instance, an “initiating entity” establishes a technique for indicating required development activities and allocating equity to those activities. Equity ownership among a plurality of developers involved in the development of a drug can therefore be readily determined in accordance with the present invention. Also, apportionment of drug revenues may be similarly accomplished as described herein.

In accordance with the present invention, equity values are assigned to different development activities. Development activities are defined herein as including any portion of the development spectrum from synthesis or collection of candidate compounds, through screening, determination of efficacy and clinical trials, to final production, regulatory approval and packaging for sale. The equity values represent a level of compensation to a contributing developer that accomplishes an identifiable development activity. Further, the amount of equity and the rate of equity distribution for a given project can vary. Factors considered in adjusting the equity in this manner include the type of drug and the scope of the particular project.

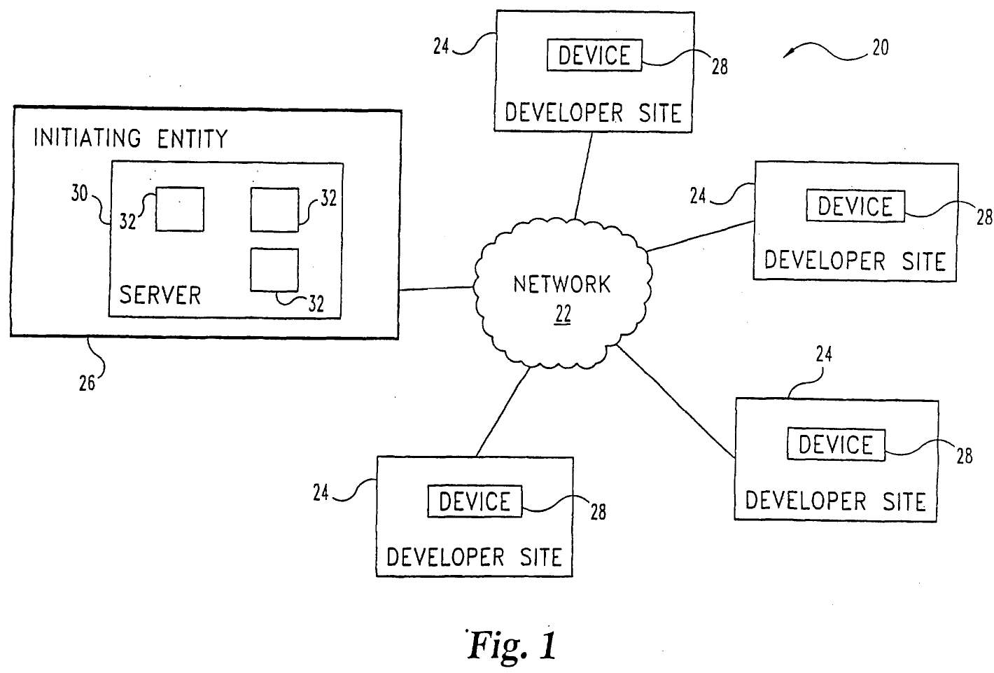

FIG. 1 illustrates development project system 20 of one embodiment of the present invention. System 20 includes computer network 22 that preferably includes a publicly accessible, Wide Area Network (WAN), such as the Internet. Network 22 communicatively links a number of developer sites 24 and project initiation/tracking site 26. Developer sites 24 each include one or more computers or other interface devices 28 to communicate over network 22. Developer sites 24 each represent one or more companies, universities, individuals, or other entities that might have an interest in participating in one or more drug development projects. While four developer sites 24 are illustrated for the sake of clarity, it should be understood that more or fewer developer sites 24 could be coupled to network 22. Typically, many more developer sites 24 would be coupled to network 22 for an embodiment of the present invention that includes the Internet.

Project initiation/tracking site 26 includes at least one computer server 30 for publishing one or more development project descriptions 32, a few of which are symbolically represented in FIG. 1. For a form of network 22 including the Internet, server 30 is preferably configured to provide a web site upon which descriptions 32 are each posted in the form of one or more web pages that can be accessed from one or more devices 28 of developer sites 24 using a standard web browser program. Alternatively or additionally, development project descriptions 32 could be published to prospective developers using different media. Indeed, in one alternative embodiment, a computer network is not included.

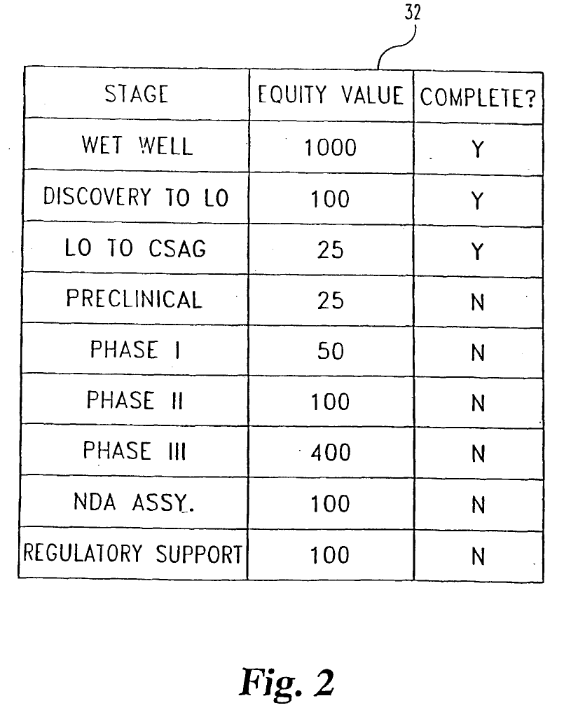

Each published development project description 32 relates to the investigation and/or development of a substance for use as a drug. Referring additionally to FIG. 2, one nonlimiting example of a development project description 32 suitable for publication as a web page with system 20 is illustrated. Description 32 is in a matrix form with three columns labeled STAGE, EQUITY VALUE, and COMPLETE? corresponding to a list of drug development project activities, assigned equity points, and activity completion status indicators, respectively. Each of these lists is organized in nine rows. The development activities for which equity values are assigned may include broad development stages. For instance, as shown in FIG. 2, evaluation of a given substance for safety and efficacy as a drug typically includes at least the following development activity stages:

1. Wet Well

2. Discovery to LO

3. LO to CSAG

4. Preclinical testing

5. Phase I Trials

6. Phase II Trials

7. Phase III Trials

8. NDA Assembly (Assy.)

9. Regulatory Support

Stage 1 corresponds to “wet well” tasks. Stage 2 is directed to discovery of a potential drug substance through Lead Optimization (LO). Stage 3, is LO through Candidate Selection Advisory Group (CSAG). In stage 4, preclinical testing usually includes initial laboratory and animal studies to assess safety and biological activity of the potential drug. Typically, it concludes with the preparation and filing of an Investigational New Drug Application (IND) with the Food and Drug Administration (FDA). Each of stages 1-4 often involve a number of different tests, evaluations, and other development activities.

Following stage 4 is a series of clinical trails required by the FDA. The phase I trial of stage 5 is the first of these trials. The phase I trial is typically performed on normal, healthy volunteers to determine a potential drug's safety profile, including a safe dosage range. The distribution, metabolization, excretion, and duration of a potential drug's action is also investigated during this stage. The phase II trial of stage 6 is directed to studies of the effectiveness of the substance for patients with the disease that the potential drug is meant to treat. In stage 7, the phase III trial is directed to further clinical studies that include more human subjects to determine efficacy and identify any adverse reactions. Typically, the clinical trials of stages 5-7 include many different sub-stages, evaluations, and other development activities.

Stage 8 relates to preparation and assembly of a New Drug Application (NDA) for submission to the FDA. Stage 9 is directed to various activities in support of FDA regulatory requirements.

Nonlimiting examples of various activities within these broad activity stages include: Structure Activity Relationship (SAR) evaluations around the lead platform; Pharmacological assay screen of SAR products; any portion of other stages, e.g., a designated portion of the clinical trials; statistical interpretation of phase III results; validated synthesis of- the potential drug substance for transfer to production; scale-up of proposed drug formulation; and transfer to manufacturing, to name just a few. Indeed, any of the development activities, stages, or phases previously described may be divided into a wide number and/or variety of sub-stages or individual actions. Frequently, two or more published development activities are performed according to a designated sequence, while the performance of other published development activities can optionally overlap, or be reversed with respect to time.

An interest in a development project posted with system 20 is assigned to a developer based on the equity value associated with the development activities completed by that developer. The equity values can be assigned to more specific development activities. As indicated, these include any identifiable portions of the development process from beginning to end. This equity value approach is arranged to entice outside developers to consider participating in the development project. In a preferred approach, the equity values are assigned as points attributable to the various activities. The proportional equity obtained by a contributing developer is determined as a proportion of the points assigned to a given activity to the total of the assigned equity points for the development process. The second column of FIG. 2 provides one nonlimiting example of an equity point allocation for the various listed development activities set forth in the first column. This allocation is arranged such that the assigned equity points for a development activity described in a given row of the first column are provided on the same row in the second column.

The distribution of equity values can differ depending on the circumstances of a given drug. For example, some of the activities may have already been accomplished or will be reserved for the initiating company. In that case, the total amount of equity available for outside developers will be correspondingly reduced. The burden or risk for certain activities will also vary depending on the drug. For example, an established drug may be developed for a new indication, and some of the development activities will therefore be simpler in view of the prior development of the drug for other purposes. Certain drugs will require more time and cost intensive clinical trials, while others will be more difficult to synthesize at production levels. FDA approvals will be more straightforward for some drugs than for others. These variations are taken into account in assigning the equity values for the development activities of various drugs.

The assigned equity values are published to potential outside developers. The publication may be in any suitable form which provides the potential developers with the information necessary to determine their desire to participate. This publication provides outside developers the opportunity to view the development activities and associated equity values. One or more developers select desired activities to be fulfilled and communicate with the initiating entity accordingly. The outside developer thereby seeks to become a “participating developer”, i.e., one which participates under the program to complete one or more development activities and to receive the indicated equity interest in the project. Such communication may take any effective form, and may preferably be accomplished by communication over a computer network, such as the Internet.

If the participating developers proceed to complete the development activities, the assigned equity values are subsequently recompensed according to a predetermined scheme. In one aspect of the invention, the equity values are reimbursed, or bought back, out of drug revenues received following launch of the drug. For this approach, the equity values determined through the point system represent fractional shares of the drug revenues, or more typically of a predetermined portion of the drug revenues, e.g., ranging from 1% to 20%. For example, in one embodiment the point fraction, e.g., 100/1000, is applied against a percentage of the drug revenues, e.g., 4%. In this example, the participating developer would receive 0.4% of the drug revenues. In a related embodiment, the equity values are reimbursed out of a limited period of drug revenues, e.g., 1 to 10 years. Therefore in this example, the participating developer would receive 0.4% of the drug revenues received during a fixed number of years following launch of the drug. The percentage and/or time period for reimbursement is determined at the time the equity values are first assigned, and will depend on the circumstances of the particular drug.

During operation of system 20, each description 32 is updated from time-to-time in response to a corresponding input to computer server 30 to reflect a change in the completion status of a given development activity. For the example of FIG. 2, completion of an activity listed in a row of the first column is indicated by a “Y” for “yes” in the third column of that same row and an activity that has not yet been completed is indicated by an “N” for “no” in the same row as the activity.

Server 30 is configured with logic to publish and track descriptions 32. This logic can be in the form of programming instructions; a hardwired, dedicated state machine; or both. In one embodiment, at least a portion of this logic is in the form of software programming instructions stored in memory associated with server 30. This memory can include one or more removable, computer-readable devices, such as floppy disks, optical disks, or the like to conveniently update and disseminate such instructions. Other data pertinent to the operation of server 30 can be stored in memory associated with server 30 as well. Server 30 is further operable to track the equity amounts assigned and status of various development project activities for each description 32. In addition, server 30 can be configured to provide data processing corresponding to the different embodiments, variations, and forms of the present invention described as follows.

In one further alternative embodiment, a development project description can additionally provide technical information pertinent to the project. This information can be in the form of one or more computer-searchable databases or computer models. In a web site-based embodiment, such information could be selectively accessed through hyperlinks and can be layered in a series of web pages to provide an interested party the option of progressively viewing more details about the project with a web browser. Computer graphics and multimedia formats could also be used to enhance the presentation. Furthermore, data obtained during the performance of various development activities by participants can be made accessible through the development project description.

While competition between different participants to complete a given development activity is preferred in certain embodiments, in an alternative preferred embodiment development activities are assigned to a given developer by a bidding procedure. In one form of this alternative, equity value is assigned based on bidding by developers. For this form, a list of the desired development activities, or “development needs”, is published to potential outside developers. The outside developers then submit bids representing the amount of equity value proposed to be received in return for completion of selected development needs. The initiating entity selects among the bids and awards the development activity to the selected participating developer. In a preferred approach, the bids are submitted over a computer network, most preferably including the Internet, and the initiating company communicates its selection of the participating companies also over the computer network.

In publishing development activities for bid, the initiating company may also publish only proposed equity values for the activities. For example, an equity value may be stated for a given development activity which is presented as a suggested value, or as a maximum value for the activity. Potential developers could choose to match the proposed value or offer a reduced value which-the developer is willing to receive as compensation for completing the development activity. Developers could also submit a bid including a request for a higher equity value. This may be premised, for example, on the belief that no other developer will accept the offered value, or in combination with other commitment terms believed to be more favorable, such as faster completion time.

Also, as an alternative to the presentation of a development project with unrestricted access, it may be desirable to publish development activities for a project with some degree of confidentiality. One approach to publishing the development needs and assigned equity values is to direct this information to selected outside developers. These outside developers may include particular persons or companies with which the initiating entity has had previous relationships, or which have known reputations regarding expertise for given tasks. In one aspect, the present invention involves a method by which development project information is provided only to entities which have already entered into a confidential agreement with the initiating company.

Alternatively, the method involves first entering into a confidential relationship with potential outside developers prior to publishing the information to those developers. One approach is to establish the confidential arrangement independently of the publication of the development needs, such as by direct contact with outside developers. In a second approach, the publication is made in a stepwise fashion. For example, a brief amount of information is provided that enables outside-developers to contact the initiating company to enter into a confidential relationship in order to obtain full information of the development needs.

In another form of the present invention, a method for an initiating entity to facilitate development of a drug through the involvement of an outside developer in at least one development activity includes: (a) the initiating entity publishing at least one development activity desired for completion by an outside developer; (b) a plurality of outside developers submitting to the initiating entity a bid to perform as a participating developer and to complete a particular development activity; (c) the initiating entity accepting one of the plurality of bids and thereby accepting the commitment of the corresponding outside developer to perform as the participating developer for the development activity; (d) each participating developer of step (c) completing at least one of the development activities; and (e) the initiating entity assigning the respective equity value in the developed drug to each participating developer according to the equity value corresponding to the development activity which the participating developer completed.

It will be appreciated that the present invention therefore provides for the development and licensing of innovative new drugs, employing coordination of worldwide scientific labs and related development facilities. Moreover, besides drugs, other products can be developed in accordance with the techniques of the present invention. For example, other products requiring a series of development phases and regulatory testing/approval, such as medical devices, could benefit from the allocation of equity to the participating developers in correspondence with various stages of the development. Participating developers realize equity in individual projects based upon the level of participation. Equity positions are bought out through profits after launch. Profitability is enhanced, and product prices can be adjusted to reflect the lower risk of distributed and diversified product development.

All publications, patents, and patent applications cited in this specification are herein incorporated by reference as if each individual publication, patent, or patent application were specifically and individually indicated to be incorporated by reference and set forth in its entirety herein. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only the preferred embodiments have been shown and described and that all changes, equivalents, and modifications that come within the spirit of the inventions defined herein and by the following claims are desired to be protected.

What is claimed is:

1. A method of medical product development, comprising:
(a) publishing a first medical product development project description, the description including a number of development activities and an equity amount for each of the development activities;
(b) communicating with an outside developer in response to said publishing;
(c) determining the outside developer completed one of the development activities; and
(d) assigning equity to the outside developer in accordance with the equity amount corresponding to the one of the development activities.

2. The method of claim 1 wherein the first medical product development project description is directed to a drug.

3. The method of claim 2 wherein the development activities correspond to one or more of the group consisting of: preclinical trial activity, phase I trials, phase II trials, phase III trials, NDA preparation; and preparation for production manufacturing of the drug.

4. The method of any of claims 1-3 further comprising communicating with a different outside developer in response to said publishing.

5. The method of claim 4 further comprising determining the different outside developer completed another of the development activities.

6. The method of claim 4 further comprising the different outside developer performing the one of the development activities.

7. The method of any of claims 1-6 further comprising publishing a second medical product development project description.

8. The method of claim 7 wherein the second medical product development project description includes a common set of the development activities relative to the first medical product development project description.

9. The method of claim 8 wherein the equity amount for the first medical product development description differs from an equity amount indicated for the second medical product development project description.

10. The method of claim 9 wherein the first medical product development project description indicates a first equity total and the second medical product development project description indicates a second equity total different than the first equity total.

11. The method of claim 9 wherein the equity amount for the first medical product development project description is allocated for the common set of the development activities differently than the equity amount indicated for the second medical product development project description.

12. The method of any of claims 1-11 wherein the outside developer belongs to a plurality of developers each performing a different one of the development activities, and further comprising making data obtained by each of the plurality of developers during performance of the different one of the development activities publicly available.

13. The method of any of claims 1-12 wherein said publishing is performed on a publicly accessible computer network.

14. The method of claim 13 wherein the publicly accessible computer network includes the internet and said publishing includes providing one or more web pages operable to be accessed with a browser program executed by a device at a remote site, the device being coupled to the network.

15. The method of any of claims 1-14 wherein the first medical product development project description includes an invitation for any interested party to perform one or more of the development activities.

16. The method of any of claims 1-14 wherein the first medical product development project description includes an indication of which of the development activities has been completed.

17. The method of claim 16 wherein the first medical product development project description includes an invitation for any interested party to perform one or more of the development activities which has not been completed.

18. A system for facilitating development of a drug, comprising:
a computer network;
a computer server coupled to the network, the computer server being programmed to present a drug development project description over the network, the drug development project description including a number of different development activities, an amount of equity points allocated to the performance of each of the different development activities, and an invitation for any interested party to perform one or more of the different development activities that has not been completed; and
wherein the computer server is responsive to an input to indicate when one of the different development activities has been completed.

19. The system of claim 18 further comprising means for presenting data related to performance of the development activities.

20. The system of claim 18 or 19 wherein the computer network includes the internet and the computer server is operable to display the drug development project description in the form of one or more web pages.

21. The system of claim 20 further comprising a plurality of devices coupled to the network, the devices each including a browser program to access the one or more web pages.

22. The system of any of claims 18-21 wherein said computer server is further operable to display a plurality of different product development project descriptions, the drug development project description belonging to said different product development project descriptions.

23. The system of claim 22 wherein the computer server maintains completion data relating to each of the different product development project descriptions.

24. The system of claim 22 or 23 wherein the computer server maintains a different equity point allocation for at least two of the different product development project descriptions.

25. A method to facilitate development of a drug, comprising:
(a) publishing a description of a development project for the drug, the description including a number of different development activities;
(b) inviting participation in the development project by any interested party;
(c) determining a first outside developer has completed one of the development activities;
(d) determining a second outside developer has completed another of the development activities; and
(e) allocating a first interest in the development project to the first outside developer and a second interest in the development project to a second outside developer.

26. The method of claim 25 wherein the first interest is in the form of an equity value in revenue produced by sales of the drug.

27. The method of claim 25 or 26 wherein the second interest is in the form of an amount of equity points, the amount of equity points being a portion of a total number of equity points allocated among the different development activities.

28. The method of any of claims 25-27 wherein the different development activities correspond to one or more of the group consisting of: preclinical trial activity, phase I trials, phase II trials, phase III trials, and NDA preparation.

29. The method of any of claims 25-28 further comprising publishing a different development project description for a different drug.

30. The method of claim 29 wherein the different drug development project description includes at least a subset of the different development activities.

31. The method of any of claims 25-30 wherein said publishing is performed on a publicly accessible computer network.

32. The method of claim 31 wherein the publicly accessible computer network includes the internet and said publishing includes providing one or more web pages operable to be accessed with a browser program executed by a device at a remote site, the device being coupled to the network.

33. The method of any of claims 25-32 further comprising updating the description to indicate which of the development activities have been completed.

34. A method for an initiating entity to facilitate development of a drug through the involvement of one or more outside developers in one or more development activities, the method comprising:
(a) the initiating entity providing a corresponding equity value for each of the one or more development activities relating to a drug being developed;
(b) the initiating entity publishing the one or more development activities each with the corresponding equity value for consideration by the one or more outside developers;
(c) the one or more outside developers communicating with the initiating entity in response to step (b);
(d) the one or more outside developers completing at least one of the development activities; and,
(e) the initiating entity assigning the corresponding equity value in the developed drug to each participating developer according to the corresponding equity value for the development activity which the participating developer completed.

35. The method of claim 34 in which said publishing comprises publishing the corresponding equity values and the one or more development activities on a computer network.

36. The method of claim 34 in which said publishing comprises transmitting the equity values and corresponding development activities to selected outside developers.

37. The method of any of claims 34-36 in which said communicating comprises communicating to the initiating entity over a computer network.

38. The method of any of claims 34-37 in which the corresponding equity values comprise interests in the sales revenues for the drug following market launch of the drug.

39. The method of claim 38 in which the assigned equity values comprise a fraction of the sales revenues for the drug.

40. The method of claim 39 and which further includes:
(f) the initiating entity compensating the participating developer based upon the equity value being applied against sales revenues for the drug after launch.

41. The method of claim 40 in which the corresponding equity value is applied against a predetermined percentage of sales revenues.

42. The method of claim 41 in which the predetermined percentage of sales revenues ranges from 1% to 20%.

43. The method of claim 40 in which the corresponding equity value is applied against the sales revenues for the drug for a predetermined period of time following launch of the drug.

44. The method of claim 43 in which said equity value fraction is applied against the annual sales revenues for the drug for a period ranging from 1 to 10 years.

45. The method of claim 34 in which the one or more outside developers number more than one and the one or more development activities number more than one and which further includes:
(f) assigning equity values to all of the development activities to be performed by the outside developers.

46. The method of claim 45 in which said assigning comprises assigning equity points to each of the development activities, the equity value for a respective one the development activities being equal to a fraction formed by dividing the equity points for the respective one development activity by the total of all equity points assigned to all the development activities.

47. The method of claim 46 and which further includes:
(g) the initiating entity compensating the participating developer based upon the equity value fraction being applied against sales revenues for the drug after launch.

48. The method of claim 47 in which the equity value fraction is applied against a predetermined percentage of sales revenues.

49. The method of claim 48 in which the predetermined percentage of sales revenues ranges from 1% to 20%.

50. The method of claim 48 in which the equity value fraction is applied against the sales revenuers for the drug for a predetermined period of time following launch of the drug.

51. The method of claim 50 in which said equity value fraction is applied against the annual sales revenues for the drug for a period ranging from 1 to 10 years.

52. The method of any of claims 34-50 in which the development activities correspond to one or more of the group consisting of preclinical trial activity, phase I trials, phase II trials, phase III trials, and NDA preparation.

53. The method of any of claims 34-52 in which price of the drug is adjusted to reflect the low risk of distributed development.

54. The method of any of claims 43-53 in which the computer network includes the internet.

55. The method of claim 54, in which said publishing includes providing one or more web pages presenting the one or more development activities each with the corresponding equity value and the one or more developers access the one or more web pages with a browser.

56. A method for an initiating entity to apportion drug revenues among a plurality of outside developers participating in activities leading to development of the drug, the method comprising:
(a) assigning equity values to the development activities performed by the participating developers;
(b) determining the equity proportion for each of the participating developers equal to the ratio of the equity value for each participating developer to the total of the equity values for all participating developers;
(c) the initiating entity compensating the participating developers based upon the equity proportion being applied against sales revenues for the drug after launch.

57. The method of claim 56 and in which further includes publishing the equity values and development activities.

58. The method of claim 57 in which said publishing includes providing one or more web pages.

59. The method of any of claims 1-17 or 25-58 wherein the development activities include at least one of a pharmacological assay screen of SAR products, a chemical synthesis task, a statistical interpretation of phase III results, and a scale-up of proposed drug formulation and transfer to manufacturing.

60. A system for assigning equity in drug revenues among a plurality of developers involved in the development of a drug, the system comprising:
(a) means for identifying several development activities associated with development of the drug for marketing; and
(b) means for assigning equity values to each of said development activities, each equity value representing an interest in the drug revenues payable to a developer participating in development of the drug.

61. A method of medical product development, comprising:
(a) receiving a published description of a medical product development project over a computer network, the description including a number of different development activities and an invitation to any interested party to perform one or more of the development activities;
(b) performing one or more of the development activities of the medical product development project in response to the invitation; and
(c) receiving equity in the development project for said performing in accordance with the published description.

62. The method of claim 61, wherein the computer network includes the internet.

63. A method of medical product development, comprising:
(a) reviewing a published description of a medical product development project from a development initiator, the description including a number of development activities and an equity amount for each of the development activities;
(b) communicating with the development initiator in response to the published description;
(c) completing one or more of the development activities; and
(d) receiving equity in accordance with the equity amount corresponding to the one or more of the development activities after said completing.

64. The method of claim 62 further comprising receiving the published description over a computer network.

65. The method of claim 64, wherein the computer network includes the internet.

66. The method of any of claims 61-65 further comprising entering a confidentiality agreement to receive additional information relating to at least one of the development activities.

67. The method of any of claims 61-66 further comprising responding to a different medical product development project description.

68. The method of any of claims 61-67 further comprising receiving data indicating which of the development activities have been completed.

69. The method of any of claims 61-68 wherein the published description is provided in the form of one or more web pages operable to be accessed with a browser program.

70. The method of any of claims 61-69 wherein the equity includes a portion of revenue from sales of a medical product developed in accordance with the published description.

71. The method of any of claims 61-70 wherein the equity is in the form of an amount of equity points, the amount of equity points being a portion of a total number of equity points allocated among the different development activities.

72. The method of any of claims 61-71 wherein the medical product development project is directed to a drug.

73. The method of any of claims 61-72 wherein the development activities correspond to one or more of the group consisting of: preclinical trial activity, phase I trials, phase II trials, phase III trials, NDA preparation, and preparation for production of a medical product.
